(19) United States
(12) Patent Application Publication
(10) Pub. No.: US 2026/0096725 A1
(43) Pub. Date: Apr. 9, 2026

(54) ON-LINE EYE PATTERN FREQUENCY MODULATION METHODS AND SYSTEMS

(71) Applicant: William V. PADULA, Killingworth, CT (US)

(72) Inventors: William V. Padula, Guilford, CT (US); Damien Gietzen, Macomb, MI (US)

(21) Appl. No.: 18/878,372

(22) PCT Filed: Jun. 27, 2023
(86) PCT No.: PCT/US2023/026309

§ 371 Date: Dec. 23, 2024

Related U.S. Application Data
(60) Provisional application No. 63/355,953, filed on Jun. 27, 2022.

(51) Int. Cl. A61B 3/032; A61B 5/00; G02B 27/01

(52) U.S. Cl. CPC A61B 3/032; A61B 5/745; G02B 27/017; G02B 2027/0174

(57) ABSTRACT

Systems and methods for delivering effective and accurate vision care diagnostics to remotely situated patients are provided. The patient/system users may be in distinct geographic locations. A diagnostic assessment may be performed by using a depth of field assessment. The chart (or computer monitor displaying a chart) and the person may move or be moved relative to each other until the focal length of the refractive error of the eye equals the distance to the chart. The refraction systems/methods can also be conducted at a specific fixed work distance instead of moving the chart(s) relative to the person or the person relative to the chart(s). In such implementations, an algorithm associated with the disclosed system/method is adjusted to calculate the refractive state of sphere, axis and cylinder power by size of the subtended angle in relationship to the distance to the chart.

### CROSS-REFERENCE TO RELATED APPLICATION

This is the U.S. National Stage application of International Application No. PCT/US2023/026309, filed Jun. 27, 2023, which claims the benefit of priority from Application No. 63/355,953, filed Jun. 27, 2022, entitled “On-line Eye Pattern Frequency Modulation Methods and Systems.” The entire contents of these prior applications are incorporated herein by reference.

### FIELD

The present disclosure is directed to methods and systems for delivering effective and accurate vision care diagnostics to remotely situated patients. The disclosed on-line eye pattern field modulation (“Oli-Eye PFM”) system/method generally involves diagnostic refraction assessment using a depth of field assessment, rather than a conventional depth of focus assessment.

### BACKGROUND

The World Health Organization (WHO) estimates that over 285 million people in the world have vision impairment and that 42% of this is due to uncorrected refractive errors. Published estimates based on epidemiological studies indicate that myopia affects 1.89 billion people worldwide and, if the current prevalence rates do not change, projections show that it will affect 2.56 billion people by 2020. Uncorrected myopia is the leading cause of vision impairment, and myopic degeneration in higher myopia was reported as the major cause of new cases of blindness in Tajimi, Japan and in Shanghai, China.

Many of the world's population live in areas where vision care is not available due to a lack of optometrists and ophthalmologists as well as the inability of the communities to afford costly examinations that include doctors requiring expensive equipment to serve patients with refractive vision care needs as well as for eye health.

Persons with disabilities and for whom it is difficult to travel/transport are often unable to benefit from ophthalmological and optometric care due to immobility.

The internet has reached most areas of the world and provides a rich source of information to communities that have limited financial resources. The internet also provides a means to reach remote populations as well as those in urban and suburban areas with limited financial means. It provides a means for the growing need for virtual medicine.

Vision exams have been developed for the internet. However, the existing refraction instruments are inaccurate screening instruments and do not protect the patient from receiving an over-prescribed prescription due to accommodative spasms causing increased pseudo-myopia. Over-prescription causes myopia to advance rapidly when care is not taken with prescriptions for children.

A need exists for improved systems and methods for providing eye-care to patients and, particularly, for delivering effective and accurate vision care diagnostics to remotely situated patients across a network, e.g., the internet. These and other needs are satisfied by the systems and methods of the present disclosure.

### BRIEF SUMMARY

The present disclosure provides advantageous system(s) and method(s) for delivering effective and accurate vision care diagnostics to remotely situated patients. As used herein, the term “remotely situated” means a patient who is not situated in the same physical location as the system user. The patient/system users may be in distinct geographic locations, e.g., different countries, different states, different cities, different physical buildings in the same city/state, or different rooms in the same building. Thus, the term “remotely situated” encompasses any situation in which the patient and the system user are not located in physical proximity to each other, i.e., they are not located in the same room such that the eye care could be provided to the patient using equipment that is located in the room with the patient and the eye care professional.

Pursuant to the disclosed on-line eye pattern field modulation (“Oli-Eye PFM”) system/method, a diagnostic assessment is performed using a depth of field assessment, rather than a conventional depth of focus assessment. Depth of focus requires that the clinician use lenses to focus light onto the fovea in the retina to improve visual acuity. With depth of field testing—as is employed according to the disclosed system/method—the chart (or computer monitor displaying a chart) and the person move or are moved relative to each other until the focal length of the refractive error of the eye equals the distance to the chart. The Oli-E PFM refraction can also be conducted at a specific fixed work distance instead of moving the chart(s) relative to the person or the person relative to the chart(s). An algorithm associated with the disclosed system/method would then be adjusted to calculate the refractive state of sphere, axis and cylinder power by size of the subtended angle in relationship to the distance to the chart.

Thus, in a first exemplary embodiment, a person/patient moves (or is moved) relative to a chart that is stationary until the focal length of the refractive error of the eye equals the distance to the chart. In a second exemplary embodiment, the chart moves/is moved relative to the person/patient who is stationary until the focal length of the refractive error of the eye equals the distance to the chart. In a third exemplary embodiment, both the person/patient and the chart are moved relative to each other until the focal length of the refractive error of the eye equals the distance to the chart. In a fourth exemplary embodiment, the linear distance from the person/patient to the chart is held constant and the angle is subtended; in such fourth exemplary embodiment, the best resolution may be projected to a preset distance, e.g., 20 feet or 6 meters, and the variance may be calculated as the refractive error of the person/patient.

In any of the first three exemplary implementations referenced above, an algorithm associated with the disclosed system/method is programmed to calculate such that the reciprocal of the focal length equals the dioptric power of the person's refractive correction.

$$D = 100 / f ( cm )$$

$$D = Diopters$$

$$f = Focal length or distance of the \\ \\ \\ person / patient from the computer monitor ( or chart )$$

In the fourth exemplary embodiment referenced above, the algorithm is adjusted so as to derive the dioptric power of the person's refractive correction based on the subtended angle.

Additional features, functions and benefits of the disclosed systems and methods will be apparent from the description which follows, particularly when read in conjunction with the accompanying figures.

### BRIEF DESCRIPTION OF THE DRAWINGS

To assist those of skill in the art in making and using the systems and methods of the present disclosure, reference is made to the accompanying figures, wherein:

FIG. 1 is a portion of a visual acuity chart that is calibrated for 6/6 (20/20) resolution;

FIG. 2 is a Padula pattern frequency system (PFS) chart according to the present disclosure;

FIG. 3 is a combination acuity/pattern frequency system (PFS) chart according to the present disclosure;

FIG. 4 is a pattern frequency system (PFS) C-star chart according to the present disclosure;

FIG. 5 is a combination bi-chrome acuity chart and pattern frequency system (PFS) chart according to the present disclosure;

FIG. 6 is a data sheet for recording from an Oli-E-PFR examination which may, in exemplary embodiments, be applied to and accessed using a cloud-based system to collect/record the data;

FIG. 7 is a graphic illustrating size of optotype acuity at a twenty foot (20′) distance with subtended discerned viewing angle;

FIG. 8 is a graphic representation of data values algorithmically employed to calculate a prescription for refractive error according to the present disclosure;

FIG. 9 is an algorithm for calculating the refractive state of sphere, axis and cylinder power by size of the subtended angle in relationship to the distance to the chart; and

FIG. 10 is a PFM or Compass Chart for use in diagnostic procedures according to the present disclosure.

### DETAILED DESCRIPTION

System(s) and method(s) for delivering effective and accurate vision care diagnostics to remotely situated patients are disclosed herein. The patient/system users may be in distinct geographic locations, e.g., different countries, different states, different cities, different physical buildings in the same city/state, or different rooms in the same building. A diagnostic assessment is performed by using a depth of field assessment. In exemplary embodiments, the chart (or computer monitor displaying a chart) and the person move or are moved relative to each other until the focal length of the refractive error of the eye equals the distance to the chart. The disclosed refraction system(s)/method(s) can also be conducted at a specific fixed work distance instead of moving the chart(s) relative to the person or the person relative to the chart(s). In such implementations, an algorithm associated with the disclosed system/method would then be adjusted to calculate the refractive state of sphere, axis and cylinder power by size of the subtended angle in relationship to the distance to the chart.

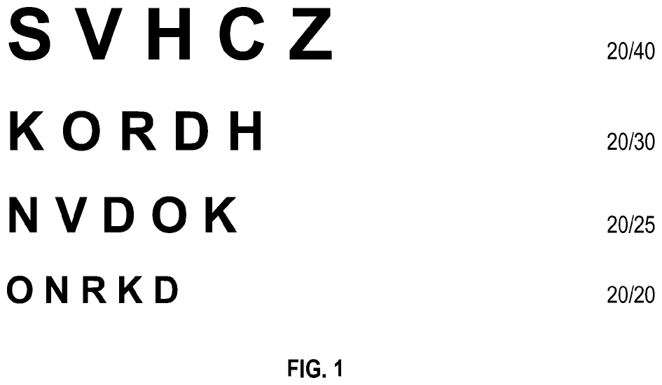

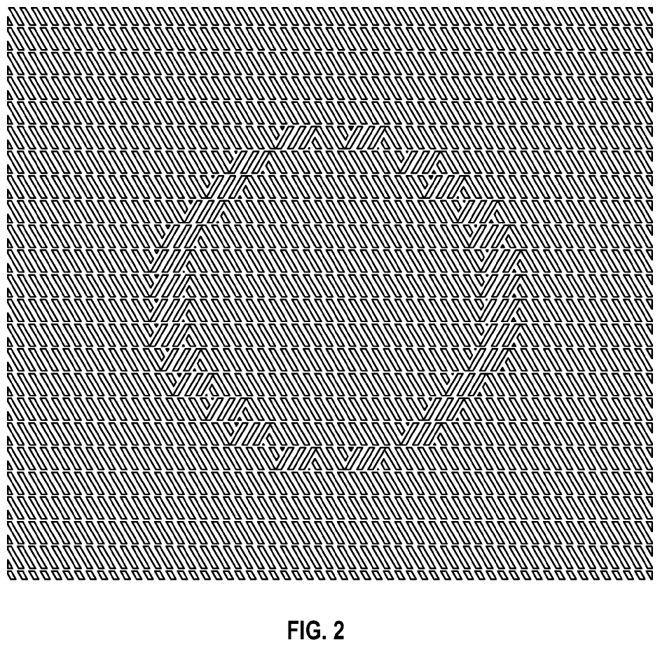

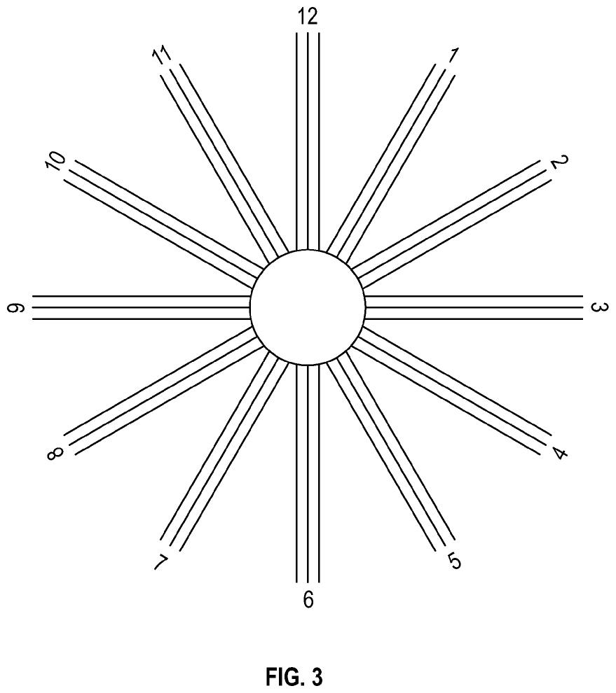

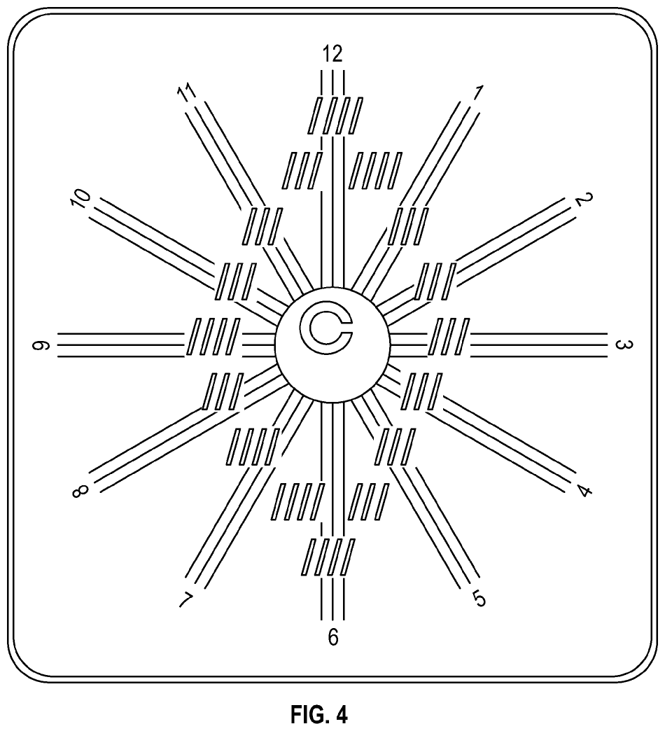

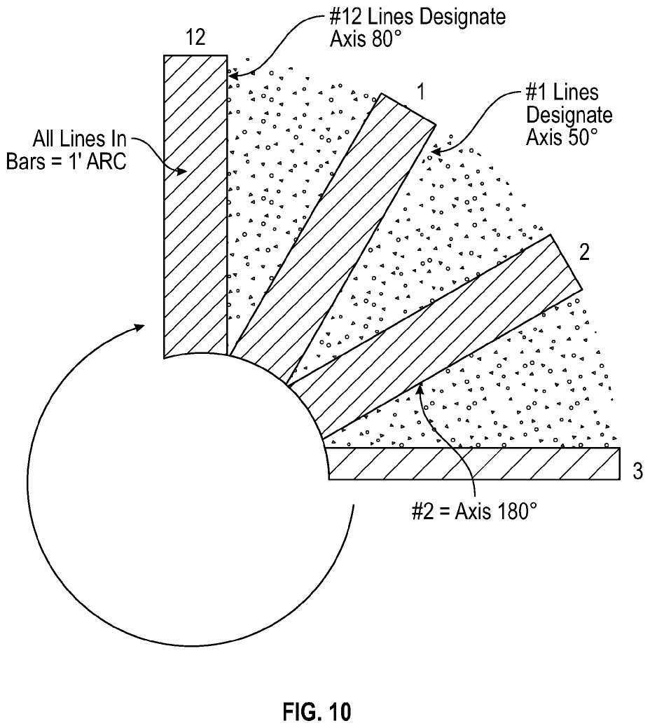

In exemplary implementations of the disclosed system/method, visual acuity charts (see FIG. 1), including the Padula Pattern Frequency System charts (PFS) (see FIG. 2), clock-dial astigmatic charts (see FIG. 3), PFS C-Star Chart (see FIG. 4) and PFM or Compass Chart (see FIG. 10) may be employed. The foregoing charts/tools enable the patient to be refracted by following instructions while looking at a computer screen/chart, even in the person's home or other remote location.

The PFS chart works with pattern receptors in the visual cortex that respond to lines and relationships of lines or patterns. This system is employed to have a response without straining to see a detail or letter. This reduces over-correction, especially in the case of myopia.

The disclosed Oli-E PFM system/method can be performed in a variety of settings, e.g., over the internet or it can be presented on a tablet or monitor in a clinical setting. In the case of the latter, a technician can present Oli-E PFM on a tablet or monitor to the patient in a hospital, clinic, or community center to provide refractions. Multiple refractions can be performed using tablets by groups of technicians to refract large numbers of patients in a short period of time.

The disclosed Oli-E PFM system/method can be cloud-based, and the prescription generated based on the disclosed vision care diagnostic procedure can be downloaded to and/or accessed by a local or distant laboratory where the lenses for each applicable prescription can be fabricated for dispensing to the patient.

The Oli-E PFM system/method can be provided on the internet using a browser/monitor or a tablet to enable the examination to be performed remotely or in a closed-circuit system presented in a clinical setting. In exemplary embodiments, the charts are designed to provide specific arc minutes of size to equal the best resolution of detail based on 1 minute of arc representing 20/20 visual acuity (6/6 metric). However, the present disclosure is not limited by or to the noted chart implementation.

Cloud-based implementations of the disclosed Oli-E PFM system/method make it possible to provide refraction to remote areas where eye and vision care services are not generally available. Due to the cost-effectiveness of the disclosed system/method, it will potentially provide refraction to those who do not have the finances to support an examination at a clinic or whose geographic location precludes refractions by a qualified professional.

The scaling of the image size has been calculated to account for magnification caused by changing working distance. Of note, the refraction requires that the image size be adapted to the size of the computer screen and the density of pixel resolution.

More specifically, the disclosed Oli-E PFM system/method is generally conducted using a series of charts presented over the internet browser on a secured website. In an exemplary implementation, to schedule for refraction testing, the patient would typically register and open an account. The registration may link the patient to a secure portal. The patient would generally need to provide demographic information as well as specifics about the following:
Type of computer monitor
Size (diagonal dimension of the screen) of the monitor screen in cm or inches
Prescription of present glasses (right and left eye)

A PIN would typically be generated and provided to the patient so that the patient could enter the secured site to conduct the testing.

### Oli-E PFM Method(s)

The Oli-E PFM system/method is generally operated/performed in the real space of a patient's environment. In an exemplary embodiment, the Oli-E PFM testing is scaled for a 6 meter (20 ft) testing distance. Any distance less than this may be re-scaled, e.g., by algorithms included within the Oli-E PFM system, so that the distance from the starting point of the exam is calibrated for the patient.

The patient is to be positioned at the 6 meter mark from the computer screen. The Oli-E PFM measures the distance of the patient to the computer screen. This can be done by several methods, such as downloading a QR code to the cell phone device of the patient. In this exemplary embodiment, the patient holds a cell phone with the QR code or a printed OR code at the level of his/her eyes or the designated device to measure the distance of the patient at the correct time of the response. If a closer distance is used, this will have been noted previously and all calculations will have been adjusted by the algorithms of the Oli-E PFM system. The patient is instructed to remove his/her habitual glasses.

In the case of presenting the disclosed Oli-E PFM system/method in a clinical setting, a technician will generally present the charts on a tablet or computer monitor and either move the instrument toward the patient or have the patient walk toward the tablet/monitor until the geometric form is observed.

### Visual Acuity

The disclosed Oli-E PFM system/method has different presentations for visual acuity and Pattern Frequency Acuity (PFA) such as:

### Visual Acuity or Pattern Frequency Acuity:

Present a standard letter chart to the patient while each eye is occluded in succession. The patient is instructed to wear his/her habitual corrective eyeglasses and to first cover their left eye. A series of slides are presented to the patient at a 6 meter distance ranging from large letters to small. The smallest line of letters that the patient can see is recorded for the habitual visual acuity. The procedure is repeated for the left eye with the right eye covered. The visual acuity can also be taken with non-literate charts, such as using the Landolt-C chart, Tumbling E Chart, or the (Padula) Pattern Frequency System (PFS) charts. The latter is incorporated into the Oli-E PFS C-Star chart (FIG. 4).

The embedded pattern frequency geometric form (PFGF) can also be used to record a PFA by presenting the PFS charts and recording the smallest angle of resolution seen by each eye. The above sequence for taking a visual acuity would be applied to the PFA.

The Oli-E PFM system/method generally uses software to provide multilingual capability so that language will not be an obstacle to providing testing and refractive care for the patient.

### Refraction

### Bi-Chrome Refractive Screening

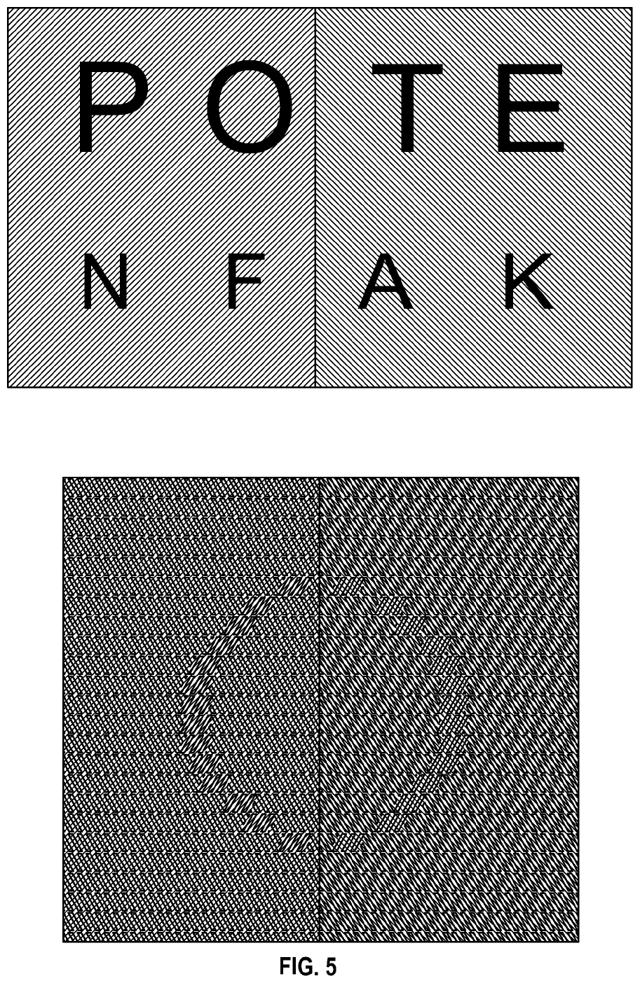

It is generally desirable to determine if a person is myopic or hyperopic will be accomplished before testing the refraction. To accomplish this task, the patient will be positioned at 6 m or an equivalent adapted testing distance from the screen. A bi-chrome acuity chart or PFS chart (half red and half green background with black letters of PFS form) will be presented monocularly to the person who either has no eyeglass prescription or with the habitual prescriptive lenses before the person's eyes (see FIG. 5). The patient will be asked on which side of the chart do the letters or the PFGF appear “blacker” or “darker”? If the response is that the red appears darker, then the person is myopic and the testing will proceed.

Due to chromatic aberration, for the emmetropic eye (refractive error equal to zero), green light focuses in front of the retina and red light focuses behind the retina. For the emmetropic eye, the red and the green side of the chart will appear equally as “black” or “dark”. For the hyperopic eye, the shift will bring the green wavelengths of light to focus on the retina causing the green to appear “blacker” or “darker” and the red side will appear lighter since the red wavelengths are moved further from the retina. For the myopic eye, the shift will be to focus the red wavelengths of light on the retina causing the red to appear “blacker” or “darker” and the green side to appear lighter.

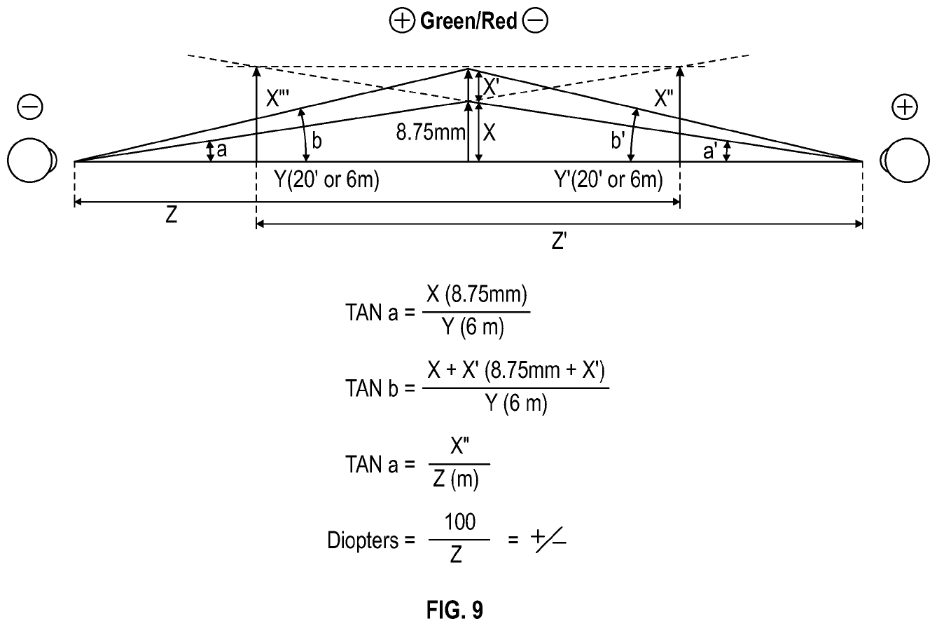

If the patient responds by saying that the green is “blacker”, then the person is hyperopic. In this case, standard plus lenses in an eyeglass frame will be introduced until the person reports that the red side is darker. FIG. 5 represents a bi-chrome acuity chart and pattern frequency system (PFS) chart that may be used for testing of a subject. In the chart of FIG. 5, the left side may be red and the right side green. If the subject reports that the lines on the green side of the chart appear blacker, the designation will be “+” and if the subject reports the lines on the red side of the chart are blacker, then a “−” sign will be designated. The testing proceeds according to the algorithms set forth in FIGS. 9 and 10. The power of the plus lenses will be recorded and the testing will continue with the patient wearing these plus lenses.

In an alternative method, the testing would proceed by providing the subject with plus lenses to essentially create a “myopic status” to the refractive error. The standard algorithms would be employed and the difference between the final refractive value minus the dioptric power of the lenses worn by the subject would be the refractive error of the spherical equivalent value of the patient's prescription. It is not necessary or required to make the hyperopic subject “myopic” to complete a diagnostic assessment based on the present disclosure (see, e.g., FIGS. 5 and 9 for bi-chrome chart/algorithmic approaches that do not require making a hyperopic subject “myopic”); rather, making a hyperopic subject “myopic” is one of the available diagnostic options.

In exemplary embodiments where the patient is hyperopic or myopic, methods/systems are disclosed wherein the patient is made “myopic”. For example, the disclosed Oli-E PFM system/method may measure the space within a 6 meter distance. In the case of the hyperope, the eye may be made pseudo-myopic in order for the focal length of the refractive error to be less than 6 m. In the case of a myope, the amount of plus lens power may be removed or added algebraically from the final prescription to yield the hyperopic refractive error for each eye.

### Oli-E PFM Refractive Sequence

In exemplary implementations of the disclosed system/method, the patient will be tested monocularly. If he/she is wearing habitual prescriptive lenses, the power of each lens must be recorded prior to testing so that this value can be added or subtracted from the final prescription of the disclosed Oli-E PFM system/method.

The patient will be positioned at the 6 meter distance (or the adjusted distance) to the computer screen/chart. Measuring the distance at which the patient first responds as seeing the PFM chart while they move toward the screen/chart or the screen/chart is moved toward the person can be accomplished in several ways:

A technician can manually record the patient responses on an interactive weblink for manual and in-patient interaction. The patient will be instructed to either move toward the screen/chart or the technician will move the screen/chart toward the patient. As soon as the patient sees the embedded PFGF, the distance is measured from the screen/chart to the patient's forehead. The distance can be measured manually, e.g., by a tape measure or other means, e.g., by a laser metric measure. This measured distance corresponds to the focal length of the dioptric power of the patient's refractive error.

An automated version of the disclosed Oli-E PFM system/method may be provided that will deliver automated directions and will use the speakers of the patient's computer or a headset to give the directions. The response by the patient will be picked up by the microphone of the computer or headset and the software of the disclosed Oli-E PFM system/method will record the response, e.g., using artificial intelligence.

In an exemplary implementation, the automated version may respond to the patient holding a QR code (e.g., printed or embedded on his/her cell phone) at the level of his/her eyes. When the patient see the PFGF, the distance may be measured automatically by the camera on the tablet or computer screen (or by other means).

Thus, the disclosed Oli-E PFM system/method is a Pattern Frequency Modulation system. Instead of using lenses to move the focal length out or into the position of the chart presented at a fixed distance, the Oli-E PFM system/method moves either the screen/chart to or from the patient or has the patient move to or from the screen/chart. Of note, the former approach is using depth of focus to shift the focal length to where the screen/chart is positioned.

The Oli-E PFM system/method uses depth of field by moving or adjusting the target on the screen/chart toward the patient or the patient toward the screen/chart. The distance of the patient from the screen/chart when the patient reports seeing the target is the focal length of the refractive error. Using the algorithm:

$$D = 100 / f ( cm )$$

The distance is then converted into the dioptric power of the correction for the refractive error of the patient.

Patients with myopia (including those having myopia and astigmatism) can be given the refraction with their habitual correction as long as the habitual prescription for each eye is known or can be measured. This habitual prescription will be added to the correction determined by the Oli-E PFM system/method. For patients that have been identified with hyperopia (including those having hyperopia and astigmatism), he/she may wear plus lenses so that the focal length of his/her uncorrected hyperopia now becomes pseudo-myopic, and the disclosed Oli-E PFM system/method may measure the refractive focal length within the 6-meter space.

For those with hyperopia, the disclosed Oli-E PFM system/method may calculate the correct hyperopic prescription by removing or adding algebraically to the power of the plus lens from the calculated dioptric measurement taken when the patient wore the additional plus lenses.

Alternatively, a diagnostic method/system is provided wherein the person or the screen need not move relative to each other. By using the size of the target at 20′ or 6 m (8.75 mm at 1′ of arc resolution for 20/20 acuity), if the subject can only remove (see) a larger size target at 20′, then the size can be used to assess the subtended angle and the focal distance of the refractive error that is designated as “+” for the hyperbole or “−” for the myope.

The patient is then instructed to cover his/her left eye and to walk slowly toward the screen/chart or the screen/chart is moved toward the patient until the PFM appears. The distance is measured, e.g., by one of the methods mentioned above. This distance is the focal length of the Spherical Equivalent refractive power of the left eye. The Spherical Equivalent is a general measurement that will account for the combination of the refractive spherical error and possible astigmatism.

The patient is then instructed to step away from the screen/chart or the clinician moves the screen/chart away from the patient until the PFGF is no longer seen. A Star Chart or the PFS Star Chart is then presented. For the Star Chart, the person is instructed to slowly move forward until one of the sets of lines corresponding to a clock dial appears “darker” or “blacker”. The patient will respond by naming the numbers of the line (e.g., 12 and 6 or) 90°. The distance to the chart/screen from the person represents the focal length of the Sphere Power. The Star Chart lines correspond to degrees of angle corresponding to the axis of astigmatism. If the person describes lines 12 and 6 as darker) (90°, this is the Cylinder Axis. The patient is then told to slowly move toward the screen or the screen is moved toward the patient until the lines that are 90° opposite the Cylinder Axis appear “blacker” or “darker”. This distance corresponds to the focal length of the Cylinder Power to correct astigmatism. The difference between the Sphere Power and the Cylinder Power equals the Minus Cylinder Refractive Error. The Cylinder Refractive Error is the difference between the first dioptric power from the first set of lines chosen (e.g., 90°) and the second set of lines chosen (180°). The procedure is repeated for the left eye. The Oli-E PFM system/method automatically converts the distance of the patient's responses as focal lengths of specific aspects of the Spherical Power, Cylinder Axis, and Cylinder Power to correct the refractive error for each eye.

The PFS-Star Chart (see FIG. 2) can advantageously be used inclusively to measure acuity, PFA, sphere power, cylinder power, and cylinder axis in the manners described above.

Alternatively, a PFM or Compass Chart as shown in FIG. 10 may be employed. The PFM Star or Compass Chart combines multiple chart functionalities into one chart. In FIG. 10, the chart is a representation where the bars of the PFM Compass chart are composed of lines with 1′ (minute of arc) separation at a specific axis. As the subject moves toward the chart, when one bar of lines appears darker and the resolution is appreciated by the subject, then this represents the sphere power as well as the axis of the astigmatism. Since the lines in the “PFM Compass” chart are at a different angle than the bar that they are imbedded in, the axis of the lines depicts that axis angle. For example, in the chart of FIG. 10, 90 degrees is represented by the bar at 2 o'clock. In use, a person moves toward the chart (or the chart is moved toward the person) until the lines in the bar 90 degrees from the original bar chosen appear blacker. The difference represents the cylinder power of the astigmatism.

In the case of using the algorithm in FIG. 9, instead of moving the chart relative to the subject or the subject relative to the chart, the target is enlarged until the appropriate lines appear blacker. The size of the target is then used to determine the range angle, and the power or refractive error is calculated without moving the subject or the chart.

### Oli-E PFM Near Refraction

A near visual acuity chart or PFM chart will be shown on the screen and the user will position themselves 16″ (40 cm) from the screen. The QR code can be used to inform the patient of the correct 40 cm working distance.

The user will respond by reading the smallest line on the acuity chart that they can read without straining or, if using the PFM chart, the patient will report the smallest chart where the geometric shape can be recognized.

This will be recorded as the uncorrected near visual acuity/PFM for each eye and both eyes together.

A bi-chrome (half green and half red background) acuity chart or PFM chart will be then presented at 40 cm. The clinician will ask the patient whether the letters on the acuity chart or the geometric form on the PFM chart appear blacker or darker on the red side or the green side. If the patient reports that the shape or letters appear darker on the green side, then the chart will be moved away until the letters of the shape appear equally dark on both the red and the green sides. The distance will be measured. This is the focal length (fy). If the red side appears darker then the chart will be moved toward the patient until equality is observed on both the red and green sides (fy). The dioptric power of fy will be calculated by:

$$D y = 100 / fy$$

The dioptric power (Dy) will be added to the sphere power algebraically to yield the add power for the bifocal:

$$Dy + Dx ( sphere power ) = Da ( + add power )$$

If the prescription is to be written as a reading lens then the Da will be algebraically added to the distance sphere power (Dx):

$$Da + Dx = Drl ( spherical diopter of reading lens )$$

For example:

If the spherical dioptric power equal −1.00 then

$$Dx = - 1.$$

If the distance the patient reports the bi-chrome chart equally black is 32.5 cm

$$100 cm / 32.5 cm = + 3 \text{.00} Dy$$

Then Dx is added algebraically with Dy to provide the add power (Da)

$$\begin{matrix}+ 3. Dy \\ \begin{matrix}\underline{- 1. Dx} \\ + 2. Da\end{matrix}\end{matrix}$$

This will conclude the Oli-E PFM refraction for distance and near ranges, and the disclosed Oli-E PFM system/method will then present the refractive correction or prescription to the examiner in the following format:

$$OD Sphere Power - Cylinder Power \times Axis / Add Power$$

$$OS Sphere Power - Cylinder Power \times Axis / Add Power$$

This PFS Star Chart can be presented with correspondence to a clock dial or it can be presented with corresponding numbers in degree axes. If the patient says that no lines appear darker, then the Spherical Equivalent becomes the Spherical (Refractive) Power correction for this eye.

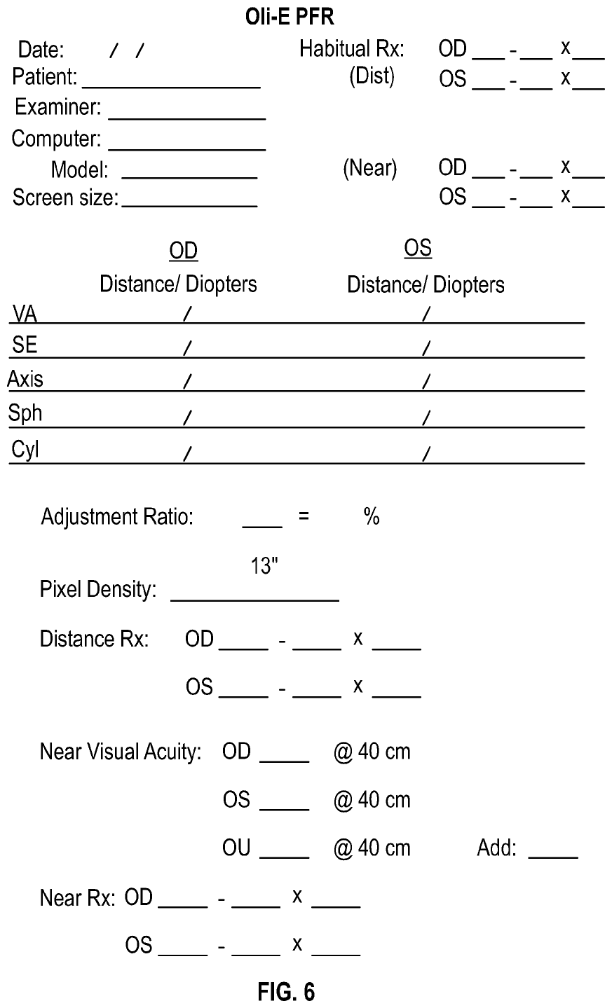

FIG. 6 provides an exemplary data sheet for collecting/recording information associated with the Oli-E PFM system/method. The data sheet can be communicated across a network (e.g., the internet) to allow data entry/capture in a remote location, and such captured/recorded data can then be accessed/used by the system and/or the practitioner to complete the diagnostic process.

The prescription can be provided to the patient at this point to have the corrective lenses fabricated or another step can be included to automatically make the lenses immediately from the prescription generated for Oli-E PFM. A generic frame in different sizes can be measured for the patient prior to or following the testing. The camera in the computer can measure the size of the patient's head and specific dimensions when the patient is at a specific distance to the computer screen. This will generate the recommended size of the frame to be used. Upon completion of the tests, the prescription is transferred to a robotic computer that is in the locale of the patient. The robotic computer will fabricate the lenses and the lenses can be inserted into the frame and given to the patient following the testing or the patient can then arrange to pick up the glasses. Another option is that the patient will be mailed the lenses. Another option is that a 3-D printer can be used to fabricate the frame for the lenses. All of these options and additional options as technology advances will reduce the cost, the time needed to test as well as fabricate the prescription.

### Oli-E PFM Near Refraction—Variation

A variation on the disclosed systems and methods for performing refraction on the Oli-E PFM refraction instrument (system/platform) is described herein. Due to the limitation of various conventional technologies (e.g., tablets, computer monitors, mobile phone displays), the disclosed method of refraction operates using a fixed distance from the monitor, rather than moving the monitor or subject (patient) closer to (or relative to) the monitor.

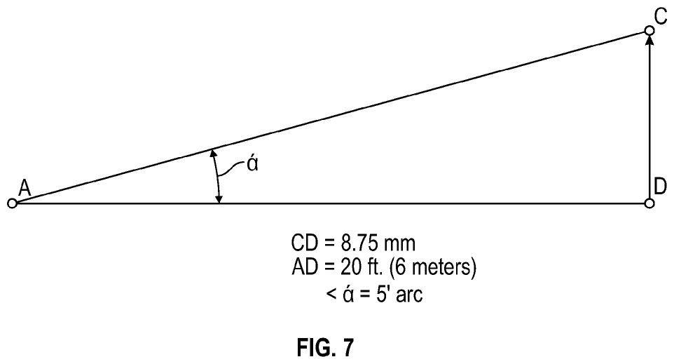

The disclosed method uses a fixed distance to the monitor. The distance to be chosen is insignificant to the efficacy of the disclosed system/method. The charts displayed, however, will need to be adjusted, adapted or designed to provide the correct subtended angle in minutes of arc. FIG. 7 shows a target (arrow) placed at 6 meters from the monitor. The subtended angle for 20/20 (6/6) acuity is 1 minute of arc separation with a 5 minute of arc optotype. This subtended angle from the monitor equals an 8.75 mm target at 20 ft. (6 meters).

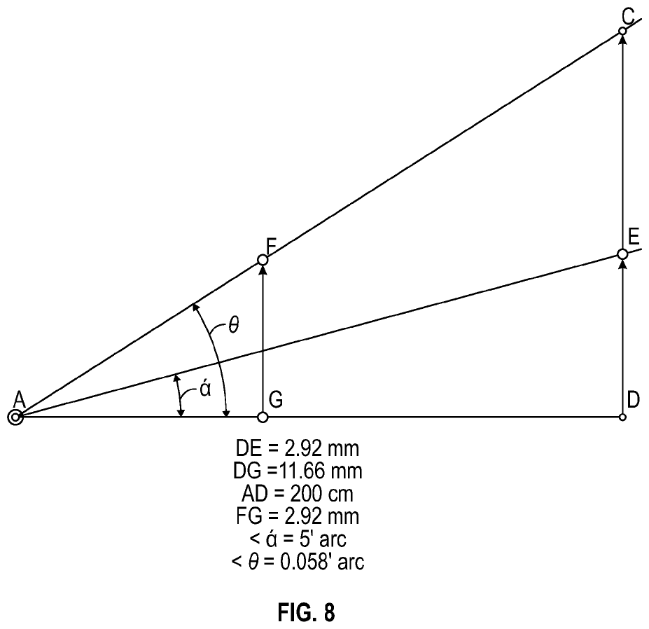

With reference to FIG. 8, two (2) meters is represented as the testing distance. This would mean that the Oli-E PFM will be presented on the monitor or screen display and that the subject will be positioned at 2 meters from the screen. At 2 meters the subject will need to accommodate-0.50 diopters. This is determined by the following formula:

$$Diopters = 100 cm / f ( cm ) = 100 / 200 = - 0.5$$

This value needs to be added to the final dioptric power.

A 20/20 optotype at 2 meters subtends the same 1 minute of arc and 5 minutes of arc subtended angle (angle β) from the screen display. The size of the 1 minute arc subtended angle within the 5 minutes of arc subtended angle is 2.92 mm.

If a subject cannot see the optotype, then the size of the optotype will be increased until it is appreciated. The larger target will subtend a greater angle. A proprietary algorithm described herein may be used to provide the distance to the subject (f=focal length of the subject's refractive error) that a 20/20 (6/6) target will need to be moved to in order for the subject to appreciate the optotype. The target does not have to be moved in space from 2 meters to accomplish this. The calculated distance to see the 20/20 target will have an angle theta (θ). The following algorithms will provide the f (focal length of the refractive error for the eye in centimeters):

$$tan {α}^{'} = \frac{Height of 20 / 20 ototype}{Distance from monitor} = \frac{2.92 cm}{200 cm} = 0 {\text{.029}}^{′} arc = \frac{11.66 cm}{200 cm} = 0 {\text{.058}}^{′} arc$$

$$0.058 = \frac{2.92 cm}{b} = 50.34 cm ( f - focal length )$$

$$\frac{100 cm}{50.34 cm} = - 2. diopters$$

$$\frac{( - ) \begin{matrix}- 2. \\ - 0.5\end{matrix}}{- 2.} Prescription for the refractive error$$

The refraction can utilize letters, numbers, and other symbols as long as they are designed to provide the appropriate subtended angle.

The Padula Pattern Frequency Modulation charts and system can also be used, causing the appearance of the geometric shape to be imbedded within an appropriately sized and designed image target. The refraction can be conducted in the same manner as discussed above with reference to the Oli-E PFM system/method.

Concerning the letters, numbers or symbols used for testing at the closer fixed distance method of refraction herein disclosed, a calculated system of size has been developed called the P-System Acuity Chart. The P-System encompasses any letters, numbers symbols or the PFM charts, geometric form, etc., that utilizes the appropriate subtended angles. The ototypes will have a mathematical relationship with the subtended angle so that the size of the chart will provide a predicted subtended angle for the subject. Enlargement of the optotype will mathematically provide a predicted subtended angle as well as acuity. Table 1 presents a representation of the acuity related to the height of the target and the focal length with dioptric equivalent.

TABLE 1
P-System Acuity-Refraction Chart

| Acuity | Height of Target (cm) | | Focal length (cm) | | Diopters |
|---|---|---|---|---|---|
| 20/20 | 2.92 | mm | 200 | cm | 0.00 |
| 20/40 | 5.83 | mm | 100 | cm | −1.00 |
| 20/80 | 11.66 | mm | 50 | cm | −2.00 |
| 20/160 | 23.33 | mm | 25 | cm | −4.00 |
| 20/320 | 46.66 | mm | 16 | cm | −6.00 |

Thus, the present disclosure provides a highly effective and efficient approach to vision care diagnostics that can be performed remotely and that relies, at least in part, on depth of field assessments. The system may be implemented using conventional electronic hardware, networks and related communication technology. In exemplary implementations, the requisite chart(s) are displayed on conventional electronic displays (e.g., laptop, phone screen, monitor). Audio communications/instructions (if any) may be delivered to speakers and/or a headset associated with the electronic device. Written communications/instructions (if any) may be delivered to the associated display. Audio feedback from the patient and/or technician (if any) may be captured by a microphone associated with the electronic device and communicated to/over the network. Similarly, written input from the patient and/or technician (if any) may be input using a keyboard/key pad associated with the electronic device. Interactions with and communications over networks, e.g., the internet, leverage conventional technology and infrastructure, as is known in the art and routinely accessed/utilized by systems/methods adapted for remote communications and interactions.

The algorithms/calculations described herein with the disclosed Oli-E PFM system/method are generally implemented through software/firmware that operates on processing unit(s) and the reference data and measured/collected data are stored in, written to and accessed from database(s), as are known in the art.

The various diagnostic tests/assessments utilizing the Oli-E PFM system/method, as described herein, are generally performed sequentially. However, the disclosed system/method does not require that all of the disclosed tests/assessments be performed for a given patient, but such tests/assessments may be performed in any combination or individually (i.e., only one test/assessment for a given patient).

The various diagnostic tests/assessments utilizing the Oli-E PFM system/method, as described herein, may be performed using a holographic eye testing device. Example embodiments provide a device for utilizing holographic virtual projection to perform eye testing, diagnosis, and prescriptive remedy.

In some embodiments, the disclosed holographic eye testing device renders on a head mounted device object(s) within the holographic display device, wherein the rendering corresponds to a virtual level of depth viewable by a user. The holographic display device may update the rendering of the object(s). The holographic display device may receive input from a user. In one embodiment, the holographic eye testing device can include a head mounted display (HMD). The HMD can include a pair of combiner lenses for rendering images within a user's field of view (FOV). The combiner lenses can be calibrated to the interpupillary distance from the user's eyes. A computing system can be connected to the combiner lenses. The HMD can be connected to an adjustable, cushioned inner headband, which can tilt the combiner lenses up and down, as well as forward and backward. To wear the unit, the user fits the HMD on his/her head, using an adjustment wheel at the back of the headband to secure it around the crown, supporting and distributing the weight of the unit equally for comfort, before tilting the visor and combiner lenses towards the front of the eyes.

The computing system can be inclusive to the HMD, where the holographic eye testing device is a self-contained apparatus. The computing system in the self-contained apparatus can include additional power circuitry to provide electrical current to the parts of the computing system. Alternatively, the computing system can be external to the HMD and communicatively coupled either through wired or wireless communication channels to the HMD. Wired communication channels can include digital video transmission formats including High Definition Multimedia Interface (HDMI), DisplayPort™ (DisplayPort is a trademark of VESA of San Jose CA, U.S.A.), or any other transmission format capable of propagating a video signal from the computing system to the combiner lenses. Additionally, the HMD can include speakers or headphones for the presentation of instructional audio to the user during the holographic eye tests. In a wireless communication embodiment, the HMD can include a wireless adapter capable of low latency high bandwidth applications, including but not limited to IEEE 802.11ad. The wireless adapter can interface with the computing system for the transmission of low latency video to be displayed upon the combiner lenses.

Additionally the computing system can include software for the manipulation and rendering of objects within a virtual space. The software can include both platform software to support any fundamental functionality of the HMD, such as motion tracking, input functionality, and eye tracking. Platform software can be implemented in a virtual reality (VR) framework, augmented reality (AR) framework, or mixed reality (MR) framework. Platform software to support the fundamental functionality can include but are not limited to SteamVR® (SteamVR is a registered trademark of the Valve Corporation, Seattle WA, U.S.A) software development kit (SDK), Oculus® VR SDK (Oculus is a registered trademark of Oculus VR LLC, Irvine CA, U.S.A.), OSVR (Open source VR) (OSVR is a registered trademark of Razer Asia Pacific Pte. Ltd. Singapore) SDK, and Microsoft Windows Mixed Reality Computing Platform. Application software executing on the computing system with the underlying platform software can be a customized rendering engine, or an off-the-shelf 3D rendering framework, such as Unity® Software (Unity Software is a registered trademark of Unity Technologies of San Francisco CA, U.S.A). The rendering framework can provide the basic building blocks of the virtualized environment for the holographic refractive eye test, including objects and manipulation techniques to change the appearance of the objects. The rendering framework can provide application programming interfaces (APIs) for the instantiation of 3D objects and well-defined interfaces for the manipulation of the 3D objects within the framework. Common software programming language bindings for rendering frameworks include but are not limited to C++, Java, and C#. Additionally, the application software can provide settings to allow a test administrator to adjust actions within the test, such as holographic object speed and object color.

Although the present disclosure has been described with reference to exemplary implementations thereof, the present disclosure is not limited by or to such exemplary implementations. Rather, various modifications, refinements, enhancements and/or improvements may be made without departing from the spirit or scope of the present disclosure. For example, as noted previously, the Oli-E PFM refraction can also be conducted at a specific fixed work distance instead of moving the chart(s) or person toward the chart. The algorithm would then be adjusted to calculate the refractive state of sphere, axis and cylinder power by size of the subtended angle in relationship to the distance to the chart. Additional modifications, refinements, enhancements and/or improvements may be made without departing from the spirit or scope of the present disclosure.

1-29. (canceled)

30. A method for performing remote visual diagnostic and/or assessment, the method comprising:
providing a chart having a target on an electronic display;
enlarging the target;
receiving an indication that an individual has noted at least one visual observation regarding the target;
upon receipt of the indication, determining a subtended angle of the target based on a size of the target and a distance of the individual to the electronic display; and
determining a diagnostic condition of the individual based on the subtended angle.

31. The method of claim 30, wherein the individual is remotely situated relative to a practitioner who is performing the visual diagnostic testing and/or assessment.

32. The method of claim 30, wherein the visual diagnostic testing and/or assessment is performed in the absence of lens-based focal length adjustment.

33. The method of claim 30, further comprising:
providing instructions for the individual to view the target of the chart and note the at least one visual observation regarding the target of the chart on the electronic display; and
providing instructions for the individual to maintain position relative to the electronic display until the indication that the individual has noted the at least one visual observation regarding the target of the chart on the electronic display is received.

34. The method of claim 30, wherein:
providing the chart is to an individual positioned a distance from the electronic display; and
enlarging the target is performed while the individual is positioned at the distance.

35. The method of claim 30, wherein the indication that the individual has noted the at least one visual observation regarding the target includes that a predefined criterion is satisfied with respect to the at least one visual observation regarding the target of the chart on the electronic display.

36. The method of claim 35, wherein enlarging the target comprises incrementally enlarging the target until the receipt of the indication that the individual has noted the at least one visual observation that satisfies the predefined criterion regarding the target.

37. The method of claim 30, wherein the chart is at least one chart selected from the group consisting of a pattern frequency chart, a combination acuity/pattern frequency system chart, a pattern frequency system C-star chart, and a combination bi-chrome acuity chart and pattern frequency system (PFS) chart.

38. The method of claim 30, wherein the diagnostic condition is selected from the group consisting of visual acuity, pattern frequency acuity, and a combination thereof.

39. A system for performing remote visual diagnostic testing, the system comprising a processor operable to run a program that:
provides a chart having a target on an electronic display;
enlarges the target;
receives an indication that an individual has noted at least one visual observation regarding the target;
upon receipt of the indication, determines a subtended angle of the target based on a size of the target and a distance of the individual to the electronic display; and
determines a diagnostic condition of the individual based on the subtended angle.

40. The system of claim 39, wherein the individual is remotely situated relative to a practitioner who is performing the visual diagnostic testing and/or assessment.

41. The system of claim 39, wherein the visual diagnostic testing and/or assessment is performed in the absence of lens-based focal length adjustment.

42. The system of claim 39, wherein the program further comprises:
providing instructions for the individual to view the target of the chart and note the at least one visual observation regarding the target of the chart on the electronic display; and
providing instructions for the individual to maintain position relative to the electronic display until the indication that the individual has noted the at least one visual observation regarding the target of the chart on the electronic display is received.

43. The system of claim 39, wherein the program:
provides the chart to an individual positioned a distance from the electronic display; and
enlarges the target while the individual is positioned at the distance.

44. The system of claim 39, wherein the indication that the individual has noted the at least one visual observation regarding the target of the chart on the electronic display includes that a predefined criterion is satisfied with respect to the at least one visual observation regarding the target of the chart on the electronic display.

45. The system of claim 44, wherein enlarging the target of the chart on the electronic display comprises incrementally enlarging the target of the chart on the electronic display until the receipt of the indication that the individual has noted the at least one visual observation that satisfies the predefined criterion regarding the target of the chart on the electronic display.

46. A method comprising conducting, remotely over a network, a diagnostic vision test on an individual positioned a fixed distance from an electronic display visible to the individual using depth of field analysis by:
(a) displaying on the electronic display a vision chart including a target image having a target image size;
(b) changing the target image size while the individual is positioned at the fixed distance;
(c) receiving, over the network, an indication when individual recognizes the target image; and
(d) calculating a refractive correction of the individual according to a subtended angle defined by the fixed distance and the target image size when the indication is received.

47. The method of claim 46, wherein the depth of field analysis is performed in the absence of lens based focal length adjustment.

48. The method of claim 46, wherein the fixed distance is measured by a distance between a patient device displaying a code and a camera associated with the electronic display and capable of seeing the code.

49. The method of claim 46, wherein the vision chart is a visual acuity chart and/or a pattern frequency chart.
